(19) United States
(12) Patent Application Publication
(10) Pub. No.: US 2023/0139543 A1
(43) Pub. Date: May 4, 2023

(54) COMPOUNDS FOR USE IN A METHOD OF TREATING, PREVENTING AND/OR REDUCING THE SYMPTOMS OF PAIN

(71) Applicant: The Beckley Foundation, Oxford (GB)

(72) Inventors: Kim KUYPERS, Maastricht (NL); Johannes G. RAMAEKERS, Maastricht (NL); Amanda FEILDING, Oxford (GB)

(21) Appl. No.: 17/905,549

(22) PCT Filed: Mar. 2, 2021
(86) PCT No.: PCT/EP2021/055120

§ 371 Date: Sep. 2, 2022

(30) Foreign Application Priority Data
Mar. 3, 2020 (GB) 2003059.9

(51) Int. Cl. A61K 31/48; A61P 25/04

(52) U.S. Cl. CPC A61K 31/48; A61P 25/04

(57) ABSTRACT

The present invention relates to compounds, i.e. lysergamides, such as lysergic acid diethylamide, for use in a method of treating (relieving), preventing and/or reducing the symptoms of pain. In some aspects, the present invention relates to a method of treating, preventing and/or reducing the symptoms of pain, which uses a lysergamide such as lysergic acid diethylamide or a pharmaceutically acceptable salt thereof.

### FIELD OF THE INVENTION

The present invention relates to compounds, namely lysergamides, such as lysergic acid diethylamide, for use in a method of treating (relieving the symptoms), preventing and/or reducing the symptoms of pain. In some aspects, the present invention relates to a method of treating, preventing and/or reducing the symptoms of pain, which uses a lysergamide such as lysergic acid diethylamide or a pharmaceutically acceptable salt thereof.

### BACKGROUND OF THE INVENTION

Pain is a distressing and unpleasant sensory experience associated with the activation of peripheral and/or central neuronal structures upon intense or damaging stimuli. Treatments to relieve pain typically include the administration of one or more analgesic drugs, which can be classified into three families: (1) primary non-opioid drugs, (2) opioid drugs, and (3) co-analgesic drugs. Non-opioid analgesic drugs include cyclooxygenase-2 (COX-2) inhibitors such as acetaminophen and non-steroidal anti-inflammatory drugs (NSAIDs).

Non-opioid analgesic drugs can be effective in treating some types of pain (e.g. light to moderate pain) but, however, may exhibit significant toxicity and/or side-effects including e.g. liver damage, ulcers, etc. Opioid drugs include natural substances such as codeine and morphine, semi-synthetic substances such as oxycodone and hydromorphone, and synthetic substances such as fentanyl. Opioid drugs are usually administered to relieve moderate to severe pain. Yet, their usefulness is limited by tolerance that rapidly develops upon treatment. Moreover, opioid drugs are highly addictive and show significant, and potentially fatal, side-effects such as respiratory depression. In 2017, more than 70,000 people died from opioid overdose in the United States (Nature Outlook: Opioids 2019, 573, 7773). Co-analgesic drugs are usually address indications other than pain, but nevertheless possess some analgesic effect for certain types of pain. Co-analgesic drugs usually show limited efficacy and may also exhibit undesirable side-effects.

Although a wide range of treatments/medications are available, pain remains a burden for millions of individuals. There is hence a need for novel treatments allowing to effectively treat, prevent and/or reduce the symptoms of pain while reducing the side-effects associated with standard pain medications.

Psychedelic substances such as mescaline, psilocybin, dimethyltryptamine (DMT) etc. have been used in traditional rites and therapy since ancient times, long before the Western world. Amongst these substances, (D)-lysergic acid diethylamide (LSD) is considered as a prototypical and reference psychedelic compound, which has been widely and successfully used by psychologists and psychiatrists in research and clinical practice until it became illegal in the mid-1960s. Recently, a considerable revival of interest has surrounded psychedelic substances including psilocybin, 5-methoxy-N,N-dimethyltryptamine (5-MeO-DMT), 2,5-dimethoxy-4-iodoamphetamine (DOI), LSD, 3,4-methylenedioxy-N-methamphetamine (MDMA), mescaline etc. in view of their considerable potential for brain research and therapy.

Clinical studies have investigated the use of psychedelics for treating psychiatric disorders including depression, existential anxiety, and substance addiction (Moreno et al. J. Clin. Psychiatry 2006, 67, 1735-1740; Grob et al. Arch. Gen. Psychiatry 2011, 68, 71-78; Johnson et al. J. Psychopharmacology 2014, 28, 983-992; Bogenschutz et al. J. Psychopharmacology 2015, 29, 289-299; Ross et al. J. Psychopharmacology 2016, 30, 1165-1180; Griffiths et al. J. Psychopharmacology 2016, 30, 1181-1197). The potential of full doses of LSD for treating pain, especially pain associated with terminal illness has also been investigated. Nonetheless, the patients may experience a profound psychedelic state, which to some is so disturbing that they refuse to continue the treatment (Whelan et al. Pain Management 2018, 8(3), 217-229; Passie et al. CNS Neur. & Ther. 2008, 14, 295-314).

In view of the foregoing, it is an object of the present invention to provide compounds for use in methods of treating, preventing and/or reducing the symptoms of pain. It is a further object of the present invention to provide pharmaceutical compositions comprising such compounds.

### SUMMARY OF THE PRESENT INVENTION

The present invention provides a compound for use in a method of treating (relieving), preventing and/or reducing the symptoms of pain comprising the administration of a lysergamide or a pharmaceutically acceptable salt thereof in an amount of 2 to 50 μg per day. Specifically, the present inventors have surprisingly found that the administration of a lysergamide such as LSD, at dose levels which are not expected to produce profound mind-altering effects (sub-hallucinogenic doses), can significantly relieve the symptoms of pain over a prolonged period of time. The present invention has been accomplished based on these findings.

The present invention thus relates to a compound for use in a method of treating, preventing and/or reducing the symptoms of pain, wherein a therapeutically effective but sub-hallucinogenic amount of a lysergamide or a pharmaceutically acceptable salt is administered to a subject.

The present invention in particular includes the following embodiments (“Items”):
1 Compound for use in a method of treating, preventing or reducing the symptoms of pain, wherein
   the compound is administered to a subject in an amount of 2 to 50 μg per day, and
   the compound is a lysergamide or a pharmaceutically acceptable salt thereof.
2. The compound for use according to item 1, wherein the compound is lysergic acid diethylamide (LSD) or a pharmaceutically acceptable salt thereof.
3. The compound for use according to item 1 or 2, wherein the compound is administered in an amount of 5 to 40 μg per day, preferably 10 to 35 μg per day, more preferably 15 to 25 μg per day, in particular 20 μg per day.
4. The compound for use according to any one of items 1 to 3, wherein the compound is administered every 1 to 6 days, preferably every 2 or 3 days, optionally over a period of up to 6 months.
5. The compound for use according to any one of items 1 to 4, wherein the pain is an acute pain or a chronic pain.
6. The compound for use according to any one of items 1 to 5, wherein the pain is selected from head pain such as cluster headache and migraine; visceral pain such as irritable bowel syndrome (IBS) and menstrual cramps; somatic pain such as postoperative pain; neuropathic pain such as fibromyalgia, central pain syndrome, complex regional pain syndrome, trigeminal neuralgia, posttraumatic neuralgia, peripheral neuropathy and herpetic/postherpetic neuralgia; inflammatory pain such as osteoarthritis, rheumatoid arthritis and atherosclerosis; functional pain such as psychogenic/psychosomatic pain and phantom limb pain; pain in advanced and progressive diseases such as pain in acquired immune deficiency syndrome (AIDS), cancer, multiple sclerosis (MS) or Crohn's disease.
7 The compound for use according to any one of items 1 to 6, wherein the pain is a neuropathic pain, preferably a neuropathic pain selected from fibromyalgia, central pain syndrome, complex regional pain syndrome, trigeminal neuralgia, posttraumatic neuralgia, peripheral neuropathy and herpetic/postherpetic neuralgia.
8. Composition for use in a method of treating, preventing or reducing the symptoms of pain, wherein the composition comprises:
   a lysergamide or a pharmaceutically acceptable salt thereof, preferably LSD or a pharmaceutically acceptable salt thereof, in an amount of 2 to 50 μg, and
   one or more components selected from a carrier, a diluent and other (pharmaceutically acceptable) excipients.
9. The compound for use according to any one of items 1 to 7 or the composition for use according to item 8, wherein the compound or the composition is administered by topical administration, parenteral administration or mucosal administration, preferably by mucosal administration such as intranasal administration, buccal administration or sublingual administration.
10. The compound for use according to any one of items 1 to 7 or the composition for use according to item 8, wherein the compound or composition is administered concurrently with, before and/or after one or more active agents selected from nonsteroidal anti-inflammatory drugs (NSAIDs), COX-2 inhibitors (COXIBs) such as acetaminophen, cannabinoids, NMDA receptor antagonists such as ketamine, magnesium, trigger point injection (TPI), and botulinum toxin.
11. The compound for use according to any one of items 1 to 7 or the composition for use according to item 8, wherein the compound or composition is administered concurrently with, before and/or after one or more selected from physiotherapy, pain management therapies such as mindfulness, guided imagery, biofeedback such as electromyographic (EMG) biofeedback, hypnosis CBT, transcutaneous electrical nerve stimulation therapy, bioelectric therapy, flotation REST therapy, chiropractic and/or osteopathic (bone) manipulation therapies, massages, visualization, acupuncture.
12. Method for treating, preventing or reducing the symptoms of pain, wherein
   a compound is administered to a subject in need thereof in an amount of 2 to 50 μg per day, and
   the compound is a lysergamide or a pharmaceutically acceptable salt thereof, preferably LSD or a pharmaceutically acceptable salt thereof.
13. The method according to item 12, wherein the method comprises administering the compound every 1 to 6 days, preferably every 2 or 3 days, optionally over a period of up to 6 months.
14. The method according to item 12 or 13, wherein the subject is a human subject.
15. The method according to any one of items 12 to 14, wherein the pain is an acute pain or a chronic pain.

### FIGURES

FIG. 1—Main (SE) pain tolerance during the Cold Pressor Test (CPT) as a function of treatment condition and time after treatment administration (*=p<0.05, relative to placebo). The administration of a low dose of LSD led to a significant increase of the pain tolerance, up to 5 hours after administration.

FIG. 2—Main (SE) painfulness during the CPT as a function of treatment condition and time after treatment administration (*=p<0.05, relative to placebo). The administration of a low dose of LSD led to a significant decrease of the painfulness, up to 5 hours after administration.

FIG. 3—Main (SE) unpleasantness during the CPT as a function of treatment condition and time after treatment administration (*=p<0.05, relative to placebo). The administration of a low dose of LSD led to a significant decrease of the pain-associated unpleasantness, up to 5 hours after administration.

FIG. 4—Main (SE) stress during the CPT as a function of treatment condition and time after treatment administration (*=p<0.05, relative to placebo). The administration of a low dose of LSD did not lead to a significant change of stress, up to 5 hours after administration.

### DETAILED DESCRIPTION OF THE PRESENT INVENTION

### 1. Definitions

The term “pain” as used herein refers to a distressing feeling often caused by intense or damaging stimuli. “Acute pain” refers to sudden pain from a specific cause (e.g. injury, infection, inflammation) which lasts for a limited period of time. “Chronic pain” refers to a persistent state of pain. Chronic pain is often associated with long-term incurable or intractable medical conditions or diseases.

Examples of pain include head pain such as cluster headache and migraine; visceral pain such as irritable bowel syndrome (IBS) and menstrual cramps; somatic pain such as postoperative pain; neuropathic pain such as fibromyalgia, central pain syndrome, complex regional pain syndrome, trigeminal neuralgia, posttraumatic neuralgia, peripheral neuropathy and herpetic/postherpetic neuralgia; inflammatory pain such as osteoarthritis, rheumatoid arthritis and atherosclerosis; functional pain such as psychogenic/psychosomatic pain and phantom limb pain; pain in advanced and progressive diseases such as pain in acquired immune deficiency syndrome (AIDS), cancer, multiple sclerosis (MS) or Crohn's disease.

The terms “treating” (or “treatment”) or “preventing” (or “prevention”) as used herein, unless otherwise indicated by context, refer to therapeutic or prophylactic treatment wherein the object is to inhibit, prevent, reduce and/or relieve the symptoms of pain. As to the present invention, beneficial or desired clinical results include e.g. alleviation of symptoms, diminishment of symptoms, stabilized (i.e. not worsening) state of pain, amelioration or palliation of the pain state, and remission (whether partial or total). Subjects in need of treatment or preventive treatment include those already experiencing pain as well as those prone to have or develop symptoms of pain.

Diagnostic tests for determining the symptoms and/or state of experienced pain are well-established in the art and known to the skilled person (e.g. the physician). For instance, the state of pain in a subject (patient) can be determined by relying on well-established diagnostic tests (e.g. a Visual Analog Scale (VAS)). Furthermore, the same diagnostic tests can also be used to determine pain progression—e.g. alleviation of pain, diminishment of extent of pain, stabilized (i.e. not worsening) state of pain, etc.—over the course of administration.

The term “therapeutically effective amount” as used herein refers to an amount of a lysergamide (e.g. LSD) effective to produce analgesia (i.e. an amount of 1 to 50 μg) and thus to treat, prevent and/or reduce the symptoms pain, but which does not induce the mind-altering effects of lysergamides. The administration of a therapeutically effective amount of the compound leads to one or more of the aforementioned beneficial or desired clinical results. Said amount refers to the dosage administered to a subject over one day (daily dosage). Said amount may nevertheless be administered to the subject in one or more sub-amounts (as determined by the physician), provided that the daily dosage is not exceeded. Furthermore, the term “therapeutically effective amount” as used herein is to be understood as the mean amount of lysergamide or pharmaceutically acceptable salt thereof administered to a subject. Accordingly, the amount of lysergamide or pharmaceutically acceptable salt thereof administered to a subject may statistically vary to define a dosage range around that mean. For instance, in some embodiments, an amount of “20 μg” is to be understood as “20 μg±1 μg”.

Lysergamides are tetracyclic molecules, ultimately derived from alkaloids produced by the ergot fungus Clavipeps Purpurea. These substances are known to exhibit agonist activity at the 5-hydroxytryptamine (5-HT_{2A}) receptor which, in the brain, plays an essential role in regulation of the cortical function and cognition. Amongst them, LSD, the diethylamide-derivative of (D)-lysergic acid reportedly constitutes one of the most potent psychedelic substances in humans.

The term “lysergamide” as used herein refers to an amide derivative of lysergic acid, preferably a compound satisfying the following formula:

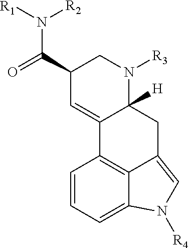

wherein R₁ and R₂ each independently represent an alkyl group having 1 to 6 carbon atoms which may be substituted, an alkene group having 1 to 6 carbon atoms which may be substituted, or an alkyne group having 1 to 6 carbon atoms which may be substituted, and R₁ and R₂ may be linked to each other to form a ring; R₃ is an alkyl group having 1 to 4 carbon atoms which may be substituted, an alkene group having 1 to 4 carbon atoms which may be substituted, or an alkyne group having 1 to 4 carbon atoms which may be substituted; and R₄ is a hydrogen atom, an alkyl group having 1 to 4 carbon atoms, or a carbonyl-containing group having 1 to 4 carbon atoms.

Examples of lysergamides include lysergic acid 2-butyl amide, lysergic acid 2-pentyl amide, lysergic acid 3-pentyl amide, lysergic acid 2-hexyl amide, N,N-diallyl lysergamide, lysergic acid methylisopropyl amide, lysergic acid diethylamide (LSD), 6-ethyl-6-nor-lysergic acid diethylamide, 6-propynyl-6-nor-lysergic acid diethylamide, 6-allyl-6-nor-lysergic acid diethylamide, 6-propyl-6-nor-lysergic acid diethylamide, 6-cyclopropyl-6-nor-lysergic acid diethylamide, 6-butyl-6-nor-lysergic acid diethylamide, 1-acetyl lysergic acid diethylamide, 1-propyonyl lysergic acid diethylamide, 1-propyonyl-6-ethyl-6-nor-lysergic acid diethylamide, N-morpholinyl lysergamide, N-pyrrolidyl lysergamide, N-piperidyl lysergamide, and lysergic acid 2,4-dimethylazetidine.

The expression “pharmaceutically acceptable salts” as used herein refers to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. Lists of suitable salts can be found in Remington's Pharmaceutical Sciences, 17th ed., Mack Publishing Company, Easton, Pa., 1985, page 1418, S. M. Berge, L. M. Bighley, and D. C. Monkhouse, “Pharmaceutical Salts,” J. Pharm. Sci. 66 (1), 1-19 (1977); P. H. Stahl and C. G. Wermuth, editors, Handbook of Pharmaceutical Salts: Properties, Selection and Use, Weinheim/Zürich, Wiley-VCH, 2008 and in A. K. Bansal et al., Pharmaceutical Technology, 3(32), 2008. The pharmaceutical salts can be synthesized from the parent compound which contains a basic or acidic moiety by conventional chemical methods. Preferably, the pharmaceutical acceptable salt is a tartrate salt.

Unless specified otherwise, chiral compounds may be present in the form of a pure stereoisomer or in the form of a mixture of stereoisomers, including the 50:50 racemate. In the context of the present invention, references to specific stereoisomers are to be understood as references to compounds, wherein the designated stereoisomer is present in at least 90% enantiomeric excess (ee), more preferably at least 95% ee and most preferably 100% ee.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, if this is technically meaningful.

### 2. Overview

The present invention is based on the finding that the administration of a lysergamide to a subject in an amount of 1 to 50 μg per day can significantly relieve the symptoms of pain, without substantially inducing the mind-altering (hallucinogenic) effects of lysergamides. It is hence expected that the compound of the present invention can be suitably and successfully used in a method of treating, preventing and/or reducing the symptoms of pain. Furthermore,

Without being bound to any theory, it is believed that the administration of a small amount lysergamide can deeply affect the subjective perception of pain as well as the objective pain tolerance, but without inducing the mind-altering effects of lysergamides. The latter is of importance, as it is expected to increase the acceptability of the drug by the patient.

### 3. Compound

The compound of the present invention is a lysergamide or a pharmaceutically acceptable salt thereof. The compound used herein (e.g. LSD) exhibits high efficacy at low doses as well as low toxicity, or no toxicity at all. Therefore, the compound of the present invention is advantageous is terms of efficacy and safety over other pain medications such as opioids.

According to one embodiment, the lysergamide is a compound satisfying the following formula:

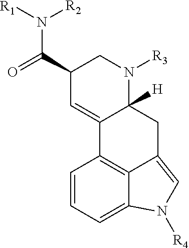

wherein R₁ and R₂ each independently represent an alkyl group having 1 to 6 carbon atoms which may be substituted, an alkene group having 1 to 6 carbon atoms which may be substituted, or an alkyne group having 1 to 6 carbon atoms which may be substituted, and R₁ and R₂ may be linked to each other to form a ring; R₃ is an alkyl group having 1 to 4 carbon atoms which may be substituted, an alkene group having 1 to 4 carbon atoms which may be substituted, or an alkyne group having 1 to 4 carbon atoms which may be substituted; and R₄ is a hydrogen atom, an alkyl group having 1 to 4 carbon atoms, or a carbonyl-containing group having 1 to 4 carbon atoms; or a pharmaceutically acceptable salt thereof.

According to one embodiment, the lysergamide is selected from lysergic acid 2-butyl amide, lysergic acid 2-pentyl amide, lysergic acid 3-pentyl amide, lysergic acid 2-hexyl amide, N,N-diallyl lysergamide, lysergic acid methylisopropyl amide, lysergic acid diethylamide (LSD), 6-ethyl-6-nor-lysergic acid diethylamide, 6-propynyl-6-nor-lysergic acid diethylamide, 6-allyl-6-nor-lysergic acid diethylamide, 6-propyl-6-nor-lysergic acid diethylamide, 6-cyclopropyl-6-nor-lysergic acid diethylamide, 6-butyl-6-nor-lysergic acid diethylamide, 1-acetyl lysergic acid diethylamide, 1-propyonyl lysergic acid diethylamide, 1-propyonyl-6-ethyl-6-nor-lysergic acid diethylamide, N-morpholinyl lysergamide, N-pyrrolidyl lysergamide, N-piperidyl lysergamide, lysergic acid 2,4-dimethylazetidine, and pharmaceutically acceptable salts thereof.

According to one preferred embodiment, the lysergamide is (D)-lysergic acid diethylamide (LSD) or a pharmaceutically acceptable salt thereof.

### 4. Composition

The compounds of the present invention can be provided in the form of pharmaceutical compositions for human or animal usage in human and veterinary medicine. Such compositions typically comprise a therapeutically effective amount (2 to 50 μg) of lysergamide according to the present invention or a pharmaceutically acceptable salt thereof, and one or more components selected from a carrier, a diluent and other (pharmaceutically acceptable) excipients. Suitable carriers, diluents and other excipients for use in pharmaceutical compositions are well known in the art, and are for instance described in Remington's Pharmaceutical Sciences, Mack Publishing Co. (Gennaro A R, 1985). The carrier, diluent and/or other excipient can be selected with regard to the intended route of administration and pharmaceutical practice. The pharmaceutical compositions can comprise as the carrier, diluents and/or other excipients, or in addition to, any suitable binder(s), lubricant(s), suspending agent(s), coating agent(s), solubilizing agent(s).

### 5. Use of Lysergamide or Composition Comprising the Same in Methods of Treating, Preventing and/or Reducing the Symptoms of Pain

The compounds of the present invention can be used to treat, prevent and/or reduce the symptoms of pain. The treatment can be a therapeutic and/or prophylactic treatment, with the aim being to prevent, reduce or stop the symptoms of pain.

The pain to be treated by the compound can be any type of pain, including chronic and acute pain which, for instance, may be associated with a pathological condition. In some aspects, the pain is selected from head pain such as cluster headache and migraine; visceral pain such as irritable bowel syndrome (IBS) and menstrual cramps; somatic pain such as postoperative pain; neuropathic pain such as fibromyalgia, central pain syndrome, complex regional pain syndrome, trigeminal neuralgia, posttraumatic neuralgia, peripheral neuropathy and herpetic/postherpetic neuralgia; inflammatory pain such as osteoarthritis, rheumatoid arthritis and atherosclerosis; functional pain such as psychogenic/psychosomatic pain and phantom limb pain; pain in advanced and progressive diseases such as pain in acquired immune deficiency syndrome (AIDS), cancer, multiple sclerosis (MS) or Crohn's disease.

According to one preferred embodiment, the pain is a neuropathic pain, more preferably a neuropathic pain selected from fibromyalgia, central pain syndrome, complex regional pain syndrome, trigeminal neuralgia, posttraumatic neuralgia, peripheral neuropathy and herpetic/postherpetic neuralgia.

According to one preferred embodiment, the subject is a human subject.

The routes for administration (delivery) include one or more of oral (e.g. tablet, capsule, ingestable solution), topical, mucosal (e.g. nasal spray, aerosol for inhalation), nasal, parenteral (e.g. an injectable form), and sublingual. According to a preferred embodiment, the compound of the present invention is administered by topical administration, parenteral administration or mucosal administration, more preferably by mucosal administration such as intranasal administration, buccal administration or sublingual administration.

The compound of the present invention is administered in an amount of 2 to 50 μg per day, preferably 5 to 40 μg per day, more preferably 10 to 35 μg per day, and most preferably 15 to 25 μg per day, in particular 20 μg per day. In one aspect, the composition of the present invention is administered such that the amount of compound (i.e. a lysergamide or a pharmaceutically acceptable salt thereof) administered is 2 to 50 μg per day, preferably 5 to 40 μg per day, more preferably 10 to 35 μg per day, and most preferably 15 to 25 μg per day, in particular 20 μg per day.

According to one preferred embodiment, the pain is neuropathic pain and the compound of the present invention is administered in an amount of 20 μg per day. According to one most preferred embodiment, the pain is neuropathic pain and the lysergamide is (D)-lysergic acid diethylamide (LSD) or a pharmaceutically acceptable salt thereof, which is administered in an amount of 20 μg per day.

According to another preferred embodiment, the compound of the present invention is administered in the aforementioned amount once (daily) every 1 to 6 days, preferably every 2 or 3 days, optionally over a period of up to 6 months, and preferably over a period of up to 60 days or up to 30 days.

In some aspects, the therapeutically effective amount can be determined by a physician on a routine basis. The specific dose level and frequency of dosage for any particular subject/patient can vary and depends on a variety of factors including the activity of the specific drug compound employed, the metabolic stability and length of action of that compound, the patient's age, body weight, general health, sex, diet, mode and time of administration, rate of excretion, drug combination, the severity of the particular condition, and the individual undergoing therapy. These factors are taken into account by the physician when determining the therapeutically effective dose.

In one embodiment, the compound (or composition) of the present invention is administered concurrently with, before and/or after (e.g. in alternance with) one or more active agents selected from nonsteroidal anti-inflammatory drugs (NSAIDs), COX-2 inhibitors (COXIBs) such as acetaminophen, cannabinoids, NMDA receptor antagonists such as ketamine, magnesium, trigger point injection (TPI), and botulinum toxin.

In another embodiment, the compound (or composition) of the present invention is administered concurrently with, before and/or after (e.g. in alternance with) one or more selected from physiotherapy, pain management therapies such as mindfulness, guided imagery, biofeedback such as electromyographic (EMG) biofeedback, hypnosis CBT, transcutaneous electrical nerve stimulation therapy, bioelectric therapy, flotation REST therapy, chiropractic and/or osteopathic (bone) manipulation therapies, massages, visualization, and acupuncture.

### 7. Examples

In the following examples, (d)-LSD (in the following simply “LSD”) dissolved in distilled water was used as the test compound. The test compound as used herein can be prepared relying on procedures and methodologies which are well-known to the person skilled in the art, e.g. on the procedure described in patent document U.S. Pat. No. 2,736,728. The test compound used in the following examples had a purity of more than 99.8% as determined by high-performance liquid chromatography (HPLC).

The effects of the test compound on the perception of pain were investigated in a placebo-controlled trial as follows:

Study Design and Treatment

Twenty-four healthy participants (12 male, 12 female) participated in a randomized, double-blind, placebo-controlled, within-subject study in which they received single oral doses of 20 μg LSD (hydrate) and placebo on 4 separate test days. A minimum washout of 5 days proceeded in between to avoid carry-over effects. Treatment orders were randomly assigned to participants according to a balanced block design. LSD was formulated as a solution of 25 μg LSD base in 1 ml 96% ethanol according to GMP and administered orally. LSD doses (0.8 ml for 20 μg LSD) were supplemented with an ethanol solution up to a total volume 1 ml and administered with a syringe under the tongue. Placebo consisted of a 1 ml ethanol solution only. Treatments were administered at 10 AM.

Participants

The mean (SD) age of participants was 22.7 (2.9) years. All participants had previous experience with psychedelics and their mean (SD) frequency of use in the year prior to the study was 2.8 (4.2) times. Reported use of psychedelics included psilocybin (N=19), LSD (N=11), DMT (N=1) and 2C-B (N=1). Other drugs that were reported included cannabis (N=23), ecstasy (N=14), amphetamines (N=7), cocaine (N=10), salvia (N=1), ketamine (N=1) and alprazolam (N=1). All participants reported the use of alcohol.

The study was conducted according to the code of ethics on human experimentation established by the declaration of Helsinki (1964) and amended in Fortaleza (Brazil, October 2013) and in accordance with the Medical Research Involving Human Subjects Act (WMO) and was approved by the Academic Hospital and University's Medical Ethics committee. All participants were fully informed about all procedures, possible adverse reactions, legal rights and responsibilities, expected benefits, and their right for voluntary termination without consequences. The study was registered in the Netherlands Trial Register (Trial NL6907 (NTR7102)).

Procedures

Participants were recruited, and candidate participants were screened and examined by a study physician, who checked for general health, conducted a resting ECG, and took blood and urine samples in which hematology, clinical chemistry, urine, and virology analyses were conducted. Inclusion criteria consisted of written informed consent, age 18-40 years, previous use of a psychedelic drug but not within the past 3 months, proficient knowledge of the English language, good physical and mental health, free from psychotropic medication, BMI between 18-28 kg/m². Exclusion criteria included history of drug abuse or addiction according to DSM-V criteria; history of psychiatric and neurological disorders, adverse response to psychedelic drugs (anxiety or panic attacks), cardiovascular abnormalities, hypertension, psychotic disorder in first-degree relatives, tobacco smoking of more than 20 cigarettes a day, excessive alcohol use (i.e. ≥20 alcohol consumptions per week), pregnancy or lactation.

Prior to the first treatment day, participants were familiarized with tests and study procedures. Participants were instructed to refrain from drug use (≥7 days) and alcohol use (≥24 hours) prior to their treatment day. They were also instructed to not consume caffeinated or alcoholic beverages on treatment days and to arrive well rested at the test facility. On arrival, participants were screened for the presence of drugs (THC, opiates, cocaine, amphetamine) in urine, and for alcohol in breath. An additional pregnancy test was given if participants were female. If all tests were found to be negative, participants were allowed to proceed.

At 1.5 and 5 hours post treatment, participants conducted a Cold Pressor Test (further described below). The Brief Symptom Inventory and the Clinician Administered Dissociation State Scale (CADSS) were administered prior to treatment administration (baseline) and at the end of a test day, i.e. at 6 hours post dosing. Vital signs were recorded at baseline, every 30 minutes during the first three hours after dosing, and at every hour thereafter. Blood samples were collected at every hour. Participants resided in a secluded room that contained a bed, table and chairs. Standardized lunches were provided around 12 AM.

The Cold Pressor Test (CPT)

The CPT was used to induce a painful sensation according to previously validated procedure (Smeets et al. Psychoneuroend. 2012, 37, 1998-2008). The CPT is the most commonly used test to induce conditioned pain modulation and to evaluate analgesic properties such as pain tolerance in healthy subjects (see, for instance, Okkerse et al. Br J Clin Pharmacol. 2017, 83(5), 976-990; Oaks et al. Discov Med. 2018, 26(144), 197-206). Furthermore, it should be noted that the evaluation of the analgesic properties of a given compound in an experimental pain model (e.g. in the CPT) is best conducted with healthy subjects as it allows for an objective efficacy assessment and a level of control, which would not be possible in a clinical pain setting, i.e. with subjects suffering from pathological pain (see, for instance, Sunil Kumar Reddy et al. Indian J Pharmacol. 2012, 44(55), 571-575).

A water tank was filled with water that was cooled to 3° C. Participants were informed that the procedure could be painful and that they could stop the task at any point without consequences. The instructions before immersion were as follows: “The aim of the task is to submerge your right hand in this cold water tank for as long as possible until you cannot take it anymore. When you cannot take it any longer, you are allowed to remove your hand from the water. Try, however, to hold on as long as possible.” The immersion duration was set to 3 minutes. Participants were not aware of this time limit. If the 3-minute maximum was achieved, the experimenter would signal the participant to remove the hand from the water. Dependent measures of the CPT included pain tolerance (sec) and subjective ratings of painfulness, unpleasantness and stress as assessed 10 cm visual analogue scales. Water temperature at onset and completion of the CPT were assessed as control measure.

Vital Signs

Heart rate (bpm) and blood pressure (mmHg) were repeatedly assessed using an Omron M6 (HEM-7321-E, Omron Healthcare Europe By) device.

Pharmacokinetics

Blood samples were centrifuged, and plasma was frozen at −20° C. until analysis for pharmacokinetic assessments. LSD plasma levels were analysed by ultra-high-performance liquid chromatography tandem mass spectrometry (UHPLC-MS/MS) as previously described previously. PK Samples with a LSD concentration below 5 μg/mL were re-analysed by a different extraction procedure. In brief, aliquots of 150 μL of plasma were extracted with 450 μL methanol. The samples were rigorously mixed and subsequently centrifuged. The supernatant was evaporated under a constant stream of nitrogen and re-suspended in 200 μL of mobile phase A and B (10:90 v/v). An LLOQ of 2.5 μg/mL was reached by this extraction.

Statistics

Analyses were carried out by means of the SPSS 25 program series to investigate whether the effects of LSD doses differed from those of placebo. CPT parameters and baseline adjusted parameters of vital signs were analysed using a three factor ANOVA to assess main effects of Treatment, Time after treatment and Participant as well as the interaction Treatment×Time after treatment. Mean contrast (LSD dose versus placebo) tests were conducted for measuring the significance of individual dose effects, relative to placebo. Canonical correlation analyses were conducted to understand the association between a set of measures of pain (i.e. pain tolerance, painfulness, unpleasantness) and a set of measures of blood pressure (systolic and diastolic blood pressure). The alpha criterion for significance was set at p<0.05.

Results

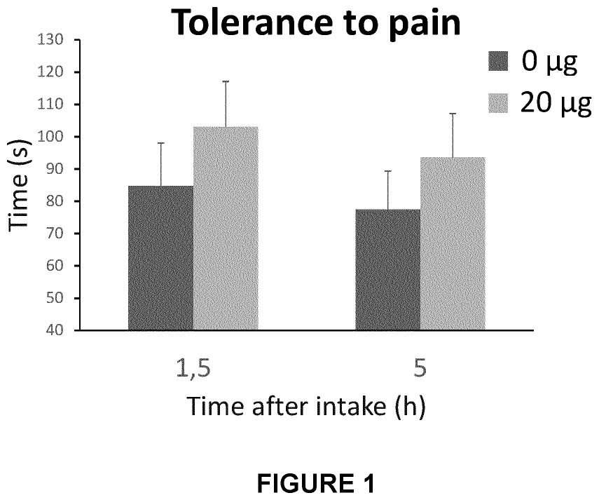

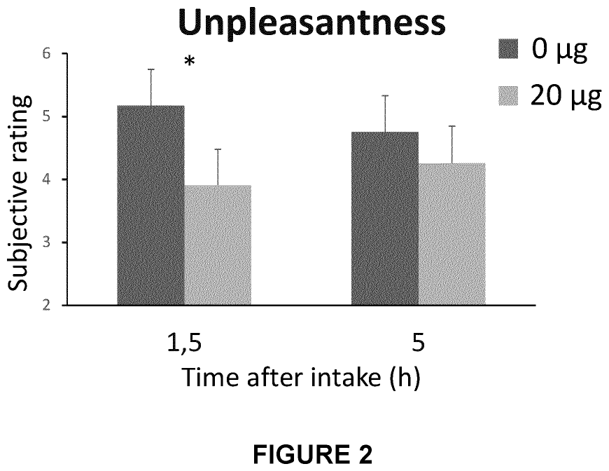

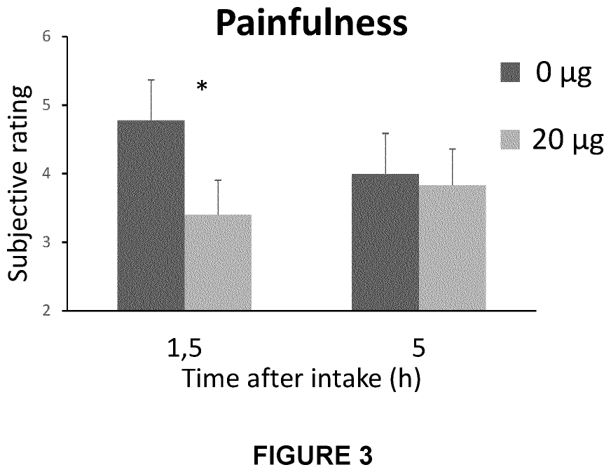

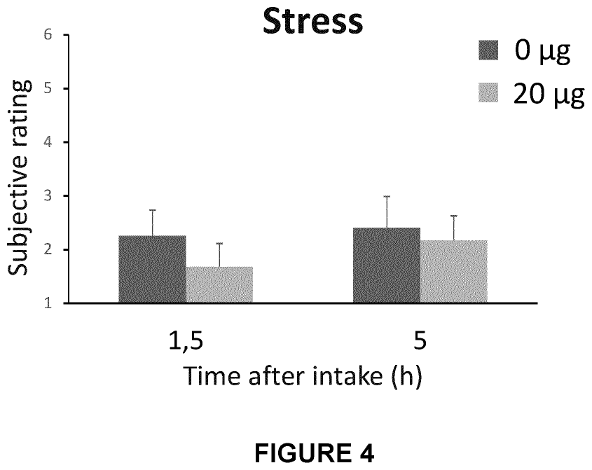

Mean (SE) pain tolerance and subjective ratings of painfulness, unpleasantness and stress during the CPT as a function of Treatment and Time after treatment administration are shown in FIGS. 1 to 4. Data analysis revealed that the administration of a low dose of LSD significantly increased pain tolerance (p=0.006) and decreased painfulness (p=0.012) as well as unpleasantness (p=0.008). Ratings of stress were not affected by Treatment, Time after treatment or their interaction. Mean (SD) water temperature at onset and end of the CPT was 2.9° C. (0.19) and 3.6° C. (0.47) and did not significantly differ between treatments and times of administration.

Mean (SE) heart rate, systolic and diastolic blood pressure as a function of treatment and time after treatment administration were also monitored (data not shown). Systolic blood pressure and diastolic blood pressure were slightly increased but nevertheless remained acceptable. Heartrate was not affected by Treatment, Time after administration or their interaction.

Pharmacokinetic analyses revealed mean (SD) plasma LSD concentrations of 482 (150) pg/mL at 1.5 hours after administration. At 6 hours post treatment, plasma LSD concentration was 223 (102) pg/mL.

1. Compound for use in a method of treating, preventing or reducing the symptoms of pain, wherein
the compound is administered to a subject in an amount of 2 to 50 μg per day, and
the compound is a lysergamide or a pharmaceutically acceptable salt thereof.

2. The compound for use according to claim 1, wherein the compound is lysergic acid diethylamide (LSD) or a pharmaceutically acceptable salt thereof.

3. The compound for use according to claim 1 or 2, wherein the compound is administered in an amount of 5 to 40 μg per day, preferably 10 to 35 μg per day, more preferably 15 to 25 μg per day, in particular 20 μg per day.

4. The compound for use according to any one of claims 1 to 3, wherein the compound is administered every 1 to 6 days, preferably every 2 or 3 days, optionally over a period of up to 6 months.

5. The compound for use according to any one of claims 1 to 4, wherein the pain is an acute pain or a chronic pain.

6. The compound for use according to any one of claims 1 to 5, wherein the pain is selected from head pain such as cluster headache and migraine; visceral pain such as irritable bowel syndrome (IBS) and menstrual cramps; somatic pain such as postoperative pain; neuropathic pain such as fibromyalgia, central pain syndrome, complex regional pain syndrome, trigeminal neuralgia, posttraumatic neuralgia, peripheral neuropathy and herpetic/postherpetic neuralgia; inflammatory pain such as osteoarthritis, rheumatoid arthritis and atherosclerosis; functional pain such as psychogenic/psychosomatic pain and phantom limb pain; and pain in advanced and progressive diseases such as pain in acquired immune deficiency syndrome (AIDS), cancer, multiple sclerosis (MS) and Crohn's disease.

7. The compound for use according to any one of claims 1 to 6, wherein the pain is a neuropathic pain, preferably a neuropathic pain selected from fibromyalgia, central pain syndrome, complex regional pain syndrome, trigeminal neuralgia, posttraumatic neuralgia, peripheral neuropathy and herpetic/postherpetic neuralgia.

8. Composition for use in a method of treating, preventing or reducing the symptoms of pain, wherein the composition comprises:
a lysergamide or a pharmaceutically acceptable salt thereof, preferably LSD or a pharmaceutically acceptable salt thereof, in an amount of 2 to 50 μg, and
one or more components selected from a carrier, a diluent and other excipients.

9. The compound for use according to any one of claims 1 to 7 or the composition for use according to claim 8, wherein the compound or the composition is administered by topical administration, parenteral administration or mucosal administration, preferably by mucosal administration such as intranasal administration, buccal administration or sublingual administration.

10. The compound for use according to any one of claims 1 to 7 or the composition for use according to claim 8, wherein the compound or composition is administered concurrently with, before and/or after one or more active agents selected from nonsteroidal anti-inflammatory drugs (NSAIDs), COX-2 inhibitors (COXIBs) such as acetaminophen, cannabinoids, NMDA receptor antagonists such as ketamine, magnesium, trigger point injection (TPI), and botulinum toxin.

11. The compound for use according to any one of claims 1 to 7 or the composition for use according to claim 8, wherein the compound or composition is administered concurrently with, before and/or after one or more selected from physiotherapy, pain management therapies such as mindfulness, guided imagery, biofeedback such as electromyographic (EMG) biofeedback, hypnosis CBT, transcutaneous electrical nerve stimulation therapy, bioelectric therapy, flotation REST therapy, chiropractic and/or osteopathic (bone) manipulation therapies, massages, visualization, acupuncture.

12. Method for treating, preventing or reducing the symptoms of pain, wherein
a compound is administered to a subject in need thereof in an amount of 2 to 50 μg per day, and
the compound is a lysergamide or a pharmaceutically acceptable salt thereof, preferably LSD or a pharmaceutically acceptable salt thereof.

13. The method according to claim 12, wherein the method comprises administering the compound every 1 to 6 days, preferably every 2 or 3 days, optionally over a period of up to 6 months.

14. The method according to claim 12 or 13, wherein the subject is a human subject.

15. The method according to any one of claims 12 to 14, wherein the pain is an acute pain or a chronic pain.
